(19) United States
(12) Patent Application Publication
(10) Pub. No.: US 2007/0239123 A1
(43) Pub. Date: Oct. 11, 2007

(54) MULTI PURPOSE CLOTH FOR USE WITH INFANTS AND CHILDREN

(76) Inventor: Margaret Webb Pressler, Washington, DC (US)

Correspondence Address: BANNER & WITCOFF, LTD., 1100 13th STREET, N.W., SUITE 1200, WASHINGTON, DC, 20005-4051, US

(21) Appl. No.: 11/278,357

(22) Filed: Mar. 31, 2006

(51) Int. Cl. A61F 13/20

(52) U.S. Cl. 604/361

(57) ABSTRACT

A multi-purpose cloth for use with infants and children is presented. The cloth includes a substantially rectangular base portion and a pocket connected to one end of the base. The pocket is configured to remain in a substantially closed position when the cloth is in a horizontal position, and a substantially open position when the cloth, or a portion of the cloth, is in a vertical position. That is, the pocket automatically falls open when the cloth is in use while burping an infant. The cloth may be made from any suitable soft, absorbent material, such as cotton, fleece and the like. Further, the cloth preferably does not include any hardware or fasteners that may harm the child or limit the use of the cloth.

### TECHNICAL FIELD

This application relates generally to a multi-purpose cloth for use with infants and young children. More particularly, this application relates to a cloth that can be used as a burp cloth for burping a child in various positions and as a multi-purpose cleaning cloth.

### BACKGROUND

As an infant is fed, gas builds within the infant and must be released. The practice of aiding the infant in releasing this gas is known as burping the infant. In order to do this, a parent or caregiver will often place the infant against his shoulder and gently pat or rub the back of the infant. This will often force the gas out of the child.

Alternative positions exist for burping an infant. In addition to burping the infant against the shoulder of the caregiver, the caregiver may also sit the child on his lap, holding the infant's head and/or chest in his hand, and gently rub or pat the back of the infant until the gas is expelled. Also, the infant can be laid across the lap of the caregiver, face down, while the caregiver rubs or pats the back of the infant until any gas is released.

Often, when gas is released, a certain amount of regurgitated liquid is released along with it. This liquid is generally regurgitated milk or formula, sometimes referred to as “spit up” or eructation. In order to protect the caregiver's clothing from being soiled by the regurgitated liquid, a cloth is often used to cover the part of the body with which the child is in contact. These cloths are typically flat, which allows any regurgitated liquid to run down the cloth onto furniture, the floor, the caregiver and the like.

### SUMMARY

This summary is provided to introduce a selection of concepts in a simplified form that are further described below in the Detailed Description. This summary is not intended to identify key features or essential features of the claimed subject matter, nor is it intended to be used as an aid in determining the scope of the claimed subject matter.

One configuration of the multi-purpose cloth for use with children includes a rectangular base portion with a pocket formed at one end. In one arrangement, the pocket may be permanently affixed to the rectangular base. The pocket is configured to remain in a substantially closed position when the cloth is in a horizontal position.

The pocket may remain in a substantially open position when the cloth is in a vertical position. The cloth may be made from a soft, absorbent material. Any substance released from an infant when being burped may be absorbed by the cloth or may be caught and retained in the pocket.

In one arrangement, the cloth may be used for burping an infant over the shoulder of an adult. The cloth may be placed over the shoulder with the pocket end covering a portion of the back of the adult. The infant may be burped facing the shoulder of the adult with the pocket in the open position to catch and retain any substance that may be released by the infant.

In another arrangement, the cloth may be used for burping an infant while sitting on the lap of an adult. The cloth may be placed over the arm of the adult with the pocket hanging in an open position. The infant may be facing the covered arm of the adult with the pocket positioned to catch and retain any substance released by the infant.

In yet another arrangement, the cloth may be used for burping an infant while lying across the lap of an adult. The cloth may be laid across the lap of the adult with the pocket hanging down in an open position. The infant may be laid across the lap, on top of the cloth, with the infant face down toward the pocket. Again the pocket is in an open position to catch and retain any substance released by the infant.

In still another arrangement, the cloth may be used to clean up general spills that a young child may cause. The absorbent cloth may be lightweight and portable for use in any such situation.

### BRIEF DESCRIPTION OF THE DRAWINGS

FIG. 1 is a top view of one configuration of a multi-purpose cloth for use with infants and children;

FIG. 2 is a side view of the multi-purpose cloth;

FIG. 3 is a top view of the multi-purpose cloth shown with the pocket in an open position;

FIG. 4 is a schematic of the multi-purpose cloth of FIG. 1;

FIG. 5 illustrates use of the multi-purpose cloth in one arrangement;

FIG. 6 illustrates use of the multi-purpose cloth according to another arrangement; and

FIG. 7 illustrates use of the multi-purpose cloth according to yet another arrangement.

### DETAILED DESCRIPTION OF THE DRAWINGS

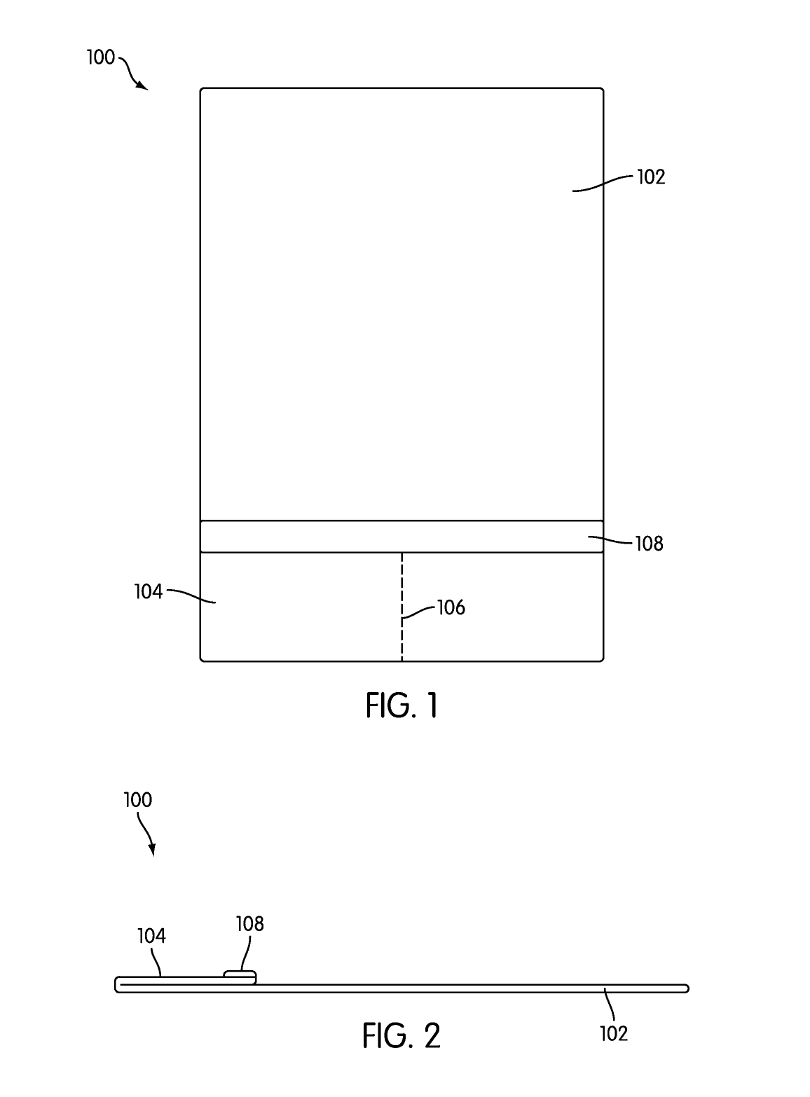

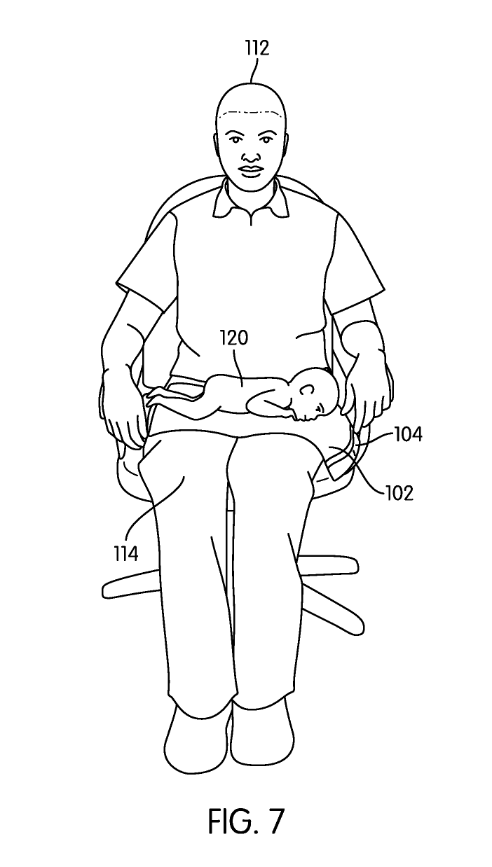

One configuration of the multi-purpose cloth for use with infants and children is the cloth 100 of FIGS. 1-7. The cloth 100 includes a rectangular portion, or base 102 with a pocket 104 formed at one end of the base 102. The pocket 104 may be configured to remain closed when the cloth 100 is in a horizontal position and open when the cloth 100 is in use in a vertical position. That is, the construction and weight of the cloth cause the pocket to automatically “fall” open when the cloth 100 is in a more vertical position (e.g., in use), and remain closed when the cloth is in a more horizontal position (e.g., lying flat or folded up).

FIGS. 1-4 depict a cloth 100 according to aspects of the multi-purpose cloth for use with infants and children. The cloth 100 may include a large, flat rectangular portion, or base 102. The base 102 may be sized to cover a portion of the body of an adult user. For example, the base 102 may be sized to cover the shoulder of a user, as well as a portion of the chest, back and upper arm. In one configuration, the rectangular base may be 12 inches wide by 18 inches long with 5 to 7 inches of the length of the base 102 covering the chest of the user. The remainder of the length of the base 102 may cover the shoulder of the user with 8 to 10 inches remaining to hang down and cover the back of the user. In another example, the base 102 may be a large enough size to cover the lap of the user, while any remaining length may hang down, covering the side of the leg of the user. The cloth 100 may be lengthened to accommodate taller or larger users, keeping in mind the pocket dimensions and/or ratios discussed below.

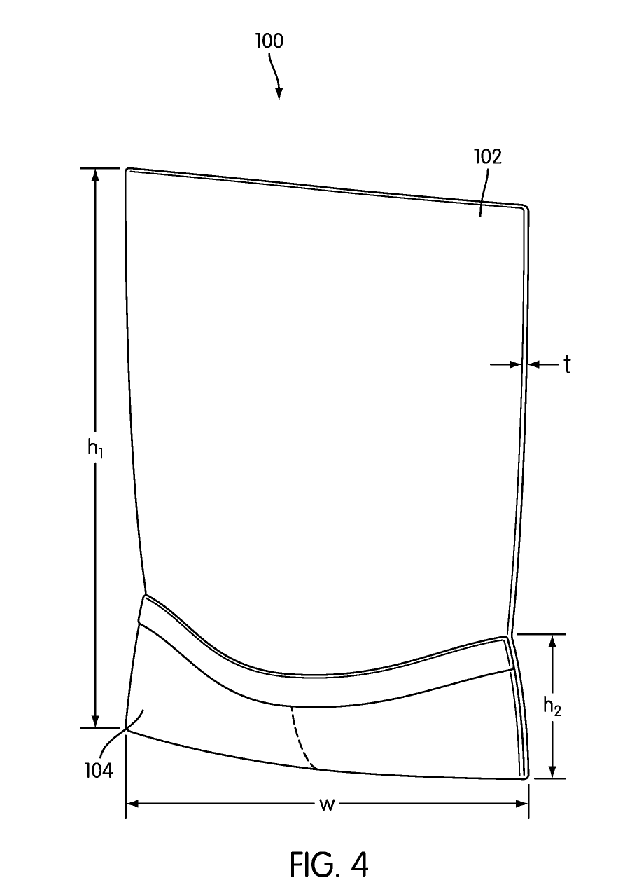

The base 102 may be formed of any suitable material that is soft and absorbent. In selecting a suitable material, attention may be paid to the proximity of the cloth to the delicate features of an infant, such as the infant's face. The material may also be lightweight and have a thickness (shown as t in FIG. 4) that allows the cloth to be easily folded to allow for portability of the cloth. The material chosen may also be washable or easily laundered. Materials that may be used can include fleece, cotton and the like. For example, the material may be flannel or a more open weave fabric.

As mentioned above, the cloth 100 may also be formed of an absorbent material. The material may be such that, as liquid contacts the cloth, the cloth traps and/or catches the liquid. The liquid may also be absorbed into the cloth. Any liquid that reaches the end of the cloth 100 may be contained within the pocket 104. The pocket 104 may also be made of the same material as the base 102 or a different material, but may also be absorbent.

The base 102 may include a pocket 104 that is formed at one end of the base 102. In one configuration, the pocket 104 may be formed by folding an end portion of a piece of material used to form base 102 over itself to a predetermined point, thereby using a single piece of material to construct both the base 102 and pocket 104. The folded portion may then be connected to the mating sides of the base 102 to form the pocket 104 that is enclosed on three sides and open at the top. Connecting the folded portion to the mating sides of the base can be by any known method of operatively coupling one portion of fabric to another. For instance, such methods can include stitching, snaps, or other forms of temporary or permanently affixing cloth pieces together. Even Velcro may be used. However, as discussed herein, it is preferable that no abrasive materials and/or components be used, so as not to accidentally harm an infant. Thus, in a preferred embodiment, only stitching is used, without the use of Velcro, snaps, or other like hardware.

In another configuration, the pocket 104 may be an additional piece of material, having the same or similar characteristics to those of the base 102. The pocket 104 of this configuration may be connected to the base 102 on three mating sides. For example, the pocket 104 may overlay an end portion of the base 102 and may be stitched along the bottom edge and the two sides of the pocket 104. This connection allows the pocket 104 to be firmly connected to the base 102, while permitting the pocket 104 to open and close freely. The connection may be alternatively accomplished by the above mentioned methods.

In one configuration, the pocket 104 and base 102 may be between 11 inches and 13 inches wide. The height of the pocket 104 may range between 5.0 inches and 5.25 inches. In one specific arrangement, the base 102 is 18 inches long, the base 102 and pocket 104 are 12 inches wide and the pocket 104 is 5.25 inches long.

FIG. 4 is a schematic of the multi-purpose cloth 100. The height of the base 102 is shown as h₁ while the height of the pocket 104 is shown as h₂. The width of the cloth 100 is labeled w. The height (h₂) and width of the pocket 104 are determined based on the height (h₁) and width of the base 102. For example, the height (h₁) to width (w) ratio may be between 0.38 and 0.47. In another example, the ratio may be 0.44. The height-to-width ratio may change, depending on the weight of the fabric used to construct cloth 100, and the dimensions and ratios provided herein are illustrative only.

Further to FIG. 1, the pocket 104 may be configured to include a seam 106. The seam 106 may be stitched in the horizontal center of the pocket 104 and extend from the top of the pocket 104 to the top of the base 102 along the back side of the base 102. The seam 106 may provide additional weight and rigidity when the pocket 104 is in an open position. The seam 106 may also aid in maintaining the open position of the pocket 104.

The pocket 104 may also include a weighted portion 108, positioned at the unattached top edge of the pocket 104. Similar to the base 102 and pocket 104, this weighted portion 108 may be formed of a delicate material to avoid harm to the infant. For example, the weighted portion 108 may be formed of an additional ribbon, piping or other type of trim that is stitched to the top edge of the pocket 104. The weighted portion 108 may also display decorative qualities that are in line with the appearance of the cloth. The weighted portion 108 may also be a portion of the material of the pocket, folded over to create a weighted hem. The weight of the weighted portion 108 aids in opening the pocket 104 when the cloth 100 is in use in a vertical position and in maintaining the open position.

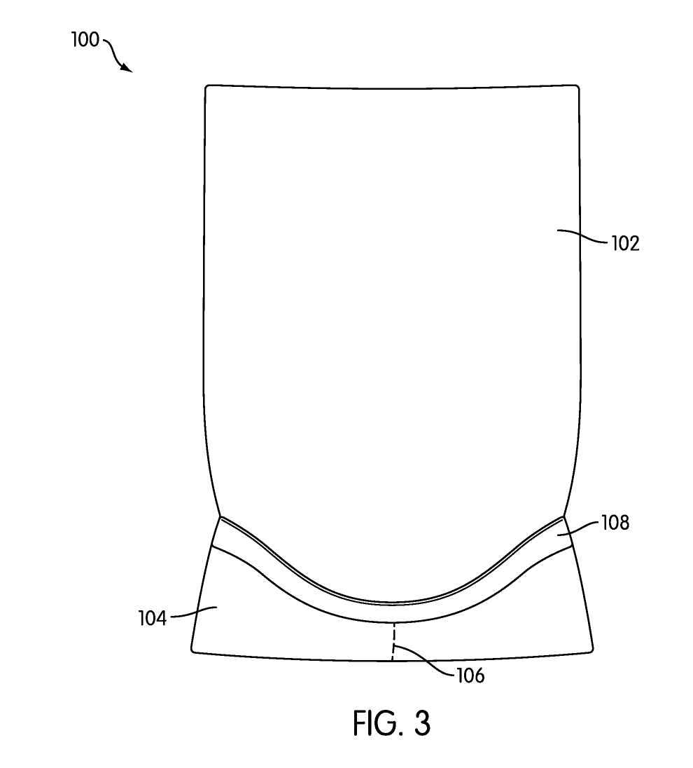

FIG. 2 illustrates the cloth 100 in a horizontal position. The pocket 104 is seen in a substantially closed position. FIG. 3 depicts the cloth 100 in a vertical position.

This vertical position causes the weighted portion 108 of the pocket 104 to fall away from the base 102, leaving the pocket 104 in a substantially open position. The pocket 104 remains in that open position, due to the weighted portion 108 and the seam 106, until the cloth 100 is returned to a horizontal position.

The multi-purpose cloth 100 preferably does not include any hardware that may scratch or injure a child. For example, the cloth 100 preferably does not require any closures, such as snaps, buttons, hook and loop closures, and the like, or weighting devices such as a block sewn into the pocket 104 or weighted portion 108 to maintain the open position. This ensures the cloth 100 is not only versatile in its many uses, but it also safe for use with small children. However, other embodiments may employ the use of child-safe hardware and/or fixtures.

In one arrangement shown in FIG. 4, the multi-purpose cloth 100 may be used for burping an infant 120. In such an arrangement, the cloth 100 may be slung over the shoulder 110 of the user 112 (e.g., an adult or other caretaker of an infant). In such an arrangement, the cloth 100 may cover a portion of the chest of the user 112, the shoulder area, as well as a portion of the back of the adult 112. This may provide substantial coverage and protection of the clothing of the user 112 if the child should spit up.

Further to FIG. 4, the pocket end of the base 102 may be in a substantially vertical configuration down the back of the user 112. This vertical configuration may cause the top weighted portion 108 to fall away from the base 102, leaving the pocket 104 in a substantially open position. The open position of the pocket allows any substance, such as regurgitated liquid, that may be released from the infant, to be caught and retained within the pocket 104.

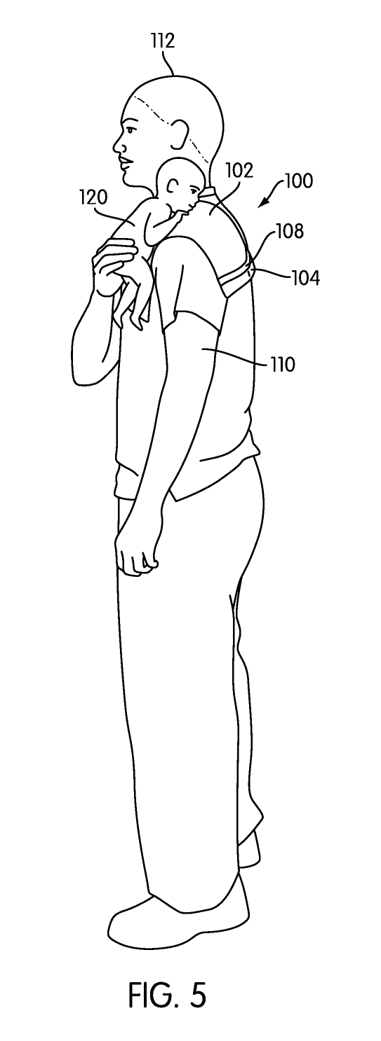

FIG. 5 illustrates another arrangement in which the multi-purpose cloth 100 may be used for burping an infant 120. In this arrangement, an infant 120 may be burped while sitting on the lap 114 of the user 112. The cloth 100 may be draped over the arm 116 of the user 112 with the pocket end of the base 102 hanging down in a vertical position. The cloth 100 may be wide enough to cover the entire forearm of the user 112. The vertical position of the base 102 allows the pocket 104 to open.

This open position allows any liquid, such as regurgitated liquid, that may be released from the infant to be caught in the pocket 104 and retained there.

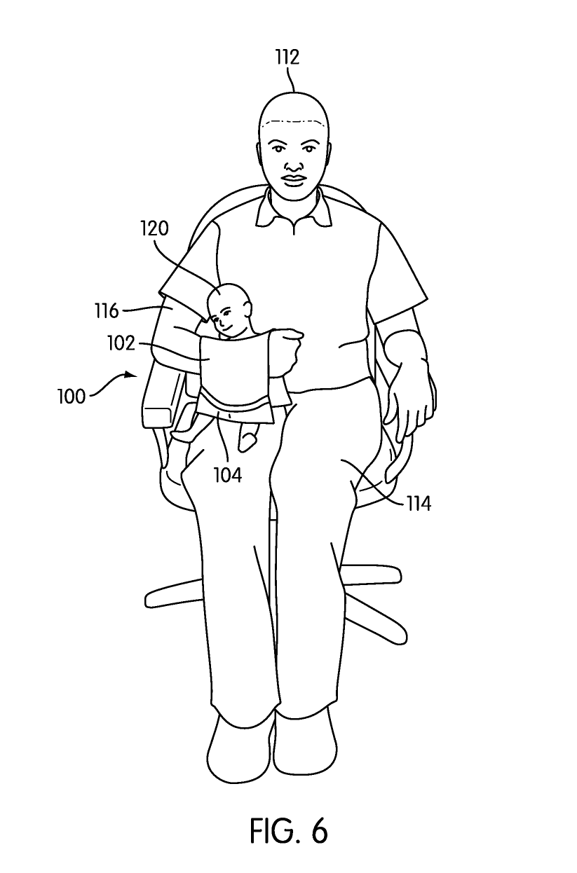

FIG. 6 illustrates yet another arrangement in which the multi-purpose cloth may be used for burping an infant. In this arrangement, an infant 120 may be burped while lying horizontally across the lap 114 of auser 112 on top of the cloth 100. The infant 120 may lay face down across the lap 114. In this arrangement, the cloth 100 may be draped across the lap 114 of the user 112, beneath the infant, with the pocket end of the base 102 hanging down in a vertical position, below the face of the infant 120.

With the cloth 100 in this vertical position, the pocket 104 may be in a substantially open position. Any substance that may be released from the infant 120 may be caught in the pocket 104 and retained therein. The absorbent material of the base 102 and the pocket 104 prevent the substance from seeping through the cloth 100.

In still another arrangement, the multi-purpose cloth 100 may be used generally for wiping up spills associated with infants and young children. The cloth 100 is lightweight and portable such that it can be folded and packed in a diaper bag to travel with the child. In addition, the absorbent material makes it useful for wiping up spills that are common with children. The use of the multi-purpose cloth 100 in this arrangement eliminates the need for a parent to carry a conventional burp cloth, as well as additional cloths for cleaning up spills.

Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims. Numerous other embodiments, modifications and variations within the scope and spirit of the appended claims will occur to persons of ordinary skill in the art from a review of this disclosure.

I claim:

1. An article of manufacture comprising:
a first substantially rectangular flat portion including a first end and a second end; and
a second substantially rectangular flat portion, configured at the first end, having three sides mating with sides of, and connected to, the first substantially rectangular flat portion, having a fourth side not connected to the first rectangular flat portion, and having a weight corresponding to the fourth side;
wherein the second substantially rectangular flat portion is configured to be flush with the first substantially rectangular flat portion when the first end is in a substantially horizontal configuration and is configured to open into a pocket when the first end is in a substantially vertical configuration.

2. The burp cloth of claim 1, wherein the first and second rectangular portions are formed of an absorbent material.

3. The burp cloth of claim 1, wherein the weight comprises a trim piece of the fourth side to maintain the open configuration of the pocket when in use.

4. The burp cloth of claim 1, wherein the first rectangular portion is sized to substantially cover the shoulder of a human user.

5. The burp cloth of claim 1, wherein the first rectangular portion is sized to cover a portion of the chest of the user.

6. The burp cloth of claim 1, wherein the first rectangular portion is sized to cover a portion of the back of the user.

7. A multi-purpose cloth, comprising:
a substantially rectangular base having a front side and a back side and including a first end and a second end, wherein the first end is folded over the base to a predetermined point on the front side and wherein the mating edges of the folded portion and the base are sewn together on the front side forming a pocket;
a weighted portion connected to the top edge of the pocket and extending between each of the sewn edges; and
a seam, the seam being stitched substantially in the horizontal center of the cloth and extending from the weighted edge of the pocket to the top of the base.

8. The multi-purpose cloth of claim 7, wherein the seam extends along the back side of the base.

9. The multi-purpose cloth of claim 7, wherein the weighted portion is a decorative element connected to the top edge of the pocket.

10. The multi-purpose cloth of claim 7, wherein the pocket is in a substantially closed position when the base is in a horizontal position and the pocket is in a substantially open position when the base is in a vertical position.

11. The multi-purpose cloth of claim 10, wherein the seam maintains the pocket in an open position.

12. The multi-purpose cloth of claim 7, wherein the base and pocket are formed of an absorbent material.

13. The multi-purpose cloth of claim 12, wherein the absorbent material is cotton.

14. The multi-purpose cloth of claim 12, wherein the absorbent material is fleece.

15. The multi-purpose cloth of claim 7, wherein the width of the base and pocket are between 11 inches and 13 inches and the height of the pocket is between 5.0 inches and 5.25 inches.

16. A method of burping an infant, comprising the steps of:
laying a multi-purpose cloth over a portion of the body of a user, wherein a first end of the multi-purpose cloth is in a substantially vertical position, and said cloth includes:
a first substantially rectangular flat portion including the first end and a second end; and
a second substantially rectangular flat portion, configured at the first end, having three sides mating with sides of, and connected to, the first substantially rectangular flat portion, having a fourth side not connected to the first rectangular flat portion, and having a weight attached to the fourth side;
wherein the second substantially rectangular flat portion is configured to open into a pocket when the first end is in a substantially vertical configuration;
placing the infant against the portion of the body that is covered by the multi-purpose cloth, wherein the infant is positioned such that the infant faces the first end with the pocket in an open position; and
patting the infant until a burp is expelled.

17. The method of claim 16, wherein the step of laying the cloth over a portion of the body includes laying the cloth over the shoulder of the user.

18. The method of claim 16, wherein the step of laying the cloth over a portion of the body includes laying the cloth over the lap of the user.

19. The method of claim 16, wherein the step of laying the cloth over a portion of the body includes laying the cloth over the arm of the user.

20. The method of claim 19, wherein the step of placing the infant includes sitting the infant on the lap of the user with the arm of the user in front of the infant.
